(19) United States
(12) Patent Application Publication
(10) Pub. No.: US 2005/0256568 A1
(43) Pub. Date: Nov. 17, 2005

(54) C-shaped heart valve prostheses

(75) Inventors: Jyue Boon Lim, Minneapolis, MN (US); William M. Sutton, Minnetonka, MN (US)

Correspondence Address: FISH & NEAVE IP GROUP;ROPES & GRAY LLP, 1251 AVENUE OF THE AMERICAS FL C3, NEW YORK, NY, 10020-1105, US

(73) Assignee: St. Jude Medical, Inc.

(21) Appl. No.: 11/126,777

(22) Filed: May 10, 2005

Related U.S. Application Data
(60) Provisional application No. 60/571,087, filed on May 14, 2004.

(51) Int. Cl. A61F 2/24

(52) U.S. Cl. 623/2.36

(57) ABSTRACT

A prosthesis for a heart valve (e.g., the mitral valve) is generally C-shaped in plan view. Points at the top and bottom of the C lie in a plan view plane. The back of the C rises above the plan view plane between the top and bottom points. Free end portions of the C may also rise above the plan view plane. The prosthesis is accordingly saddle-shaped. The back of the C may have an indentation that extends toward the open side of the C. In use as a mitral valve prosthesis the top and bottom of the C are respectively adjacent the commissures of the valve, and the back of the C is adjacent the posterior section of the valve. The prosthesis may be rigid or semi-rigid.

This application claims the benefit of U.S. provisional patent application No. 60/571,087, filed May 14, 2004, which is hereby incorporated by reference herein in its entirety.

### BACKGROUND OF THE INVENTION

Annuloplasty rings are well known as prostheses for heart valves that are not functioning properly. See, for example, Alfieri et al. U.S. patent application publication U.S. 2002/0173844 A1 and Bolling et al. U.S. patent application publication U.S. 2003/0093148 A1. Prostheses that are less than complete rings are also known for this purpose. See, for example, Carpentier U.S. Pat. No. 3,656,185. The less-than-complete rings that are known tend to be flat. This may not be the best shape for providing the most effective and beneficial prosthesis. This invention aims at providing less-than-complete-ring prostheses having more effective shapes and other beneficial features.

### SUMMARY OF THE INVENTION

A heart valve prosthesis in accordance with the invention is generally C-shaped in plan view. Points at the top and bottom of the C lie in a plan view plane. The back of the C rises above the plan view plane between the top and bottom points. Free end portions of the C (remote from the back, beyond the top and bottom points) may also rise above the plan view plane. The prosthesis is accordingly preferably saddle-shaped. The back of the C may also have an indentation or pinch that extends inwardly toward the open side of the C. In use as a mitral valve prosthesis, for example, the top and bottom of the C are respectively adjacent the commissures of the valve, and the back of the C is adjacent the posterior section of the valve. The prosthesis may be rigid or semi-rigid.

Further features of the invention, its nature and various advantages, will be more apparent from the accompanying drawings and the following detailed description of the preferred embodiments.

### BRIEF DESCRIPTION OF THE DRAWINGS

FIG. 1 is a simplified plan view of an illustrative embodiment of a heart valve prosthesis in accordance with the invention.

FIG. 2 is a simplified elevational view taken along the line 2-2 in FIG. 1.

FIG. 3 is another simplified elevational view taken along the line 3-3 in FIG. 2.

FIG. 4 is another view similar to FIG. 1 with some dimensional references added.

FIG. 5 is similar to FIG. 1, but shows another illustrative embodiment in accordance with the invention.

FIG. 6 is a simplified elevational view taken along the line 6-6 in FIG. 5.

### DETAILED DESCRIPTION

An illustrative embodiment of a heart valve prosthesis 10 in accordance with the invention is shown in FIGS. 1-4. This illustrative embodiment is intended for use as a mitral valve prosthesis. In that application prosthesis 10 will be implanted part way around a patient's mitral valve, with the posterior portion of the valve on the left as viewed in FIG. 1.

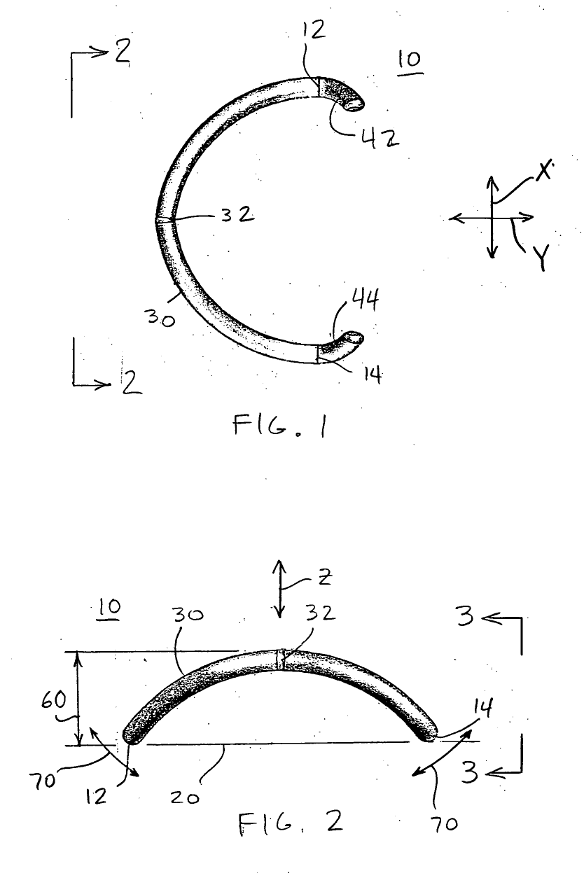

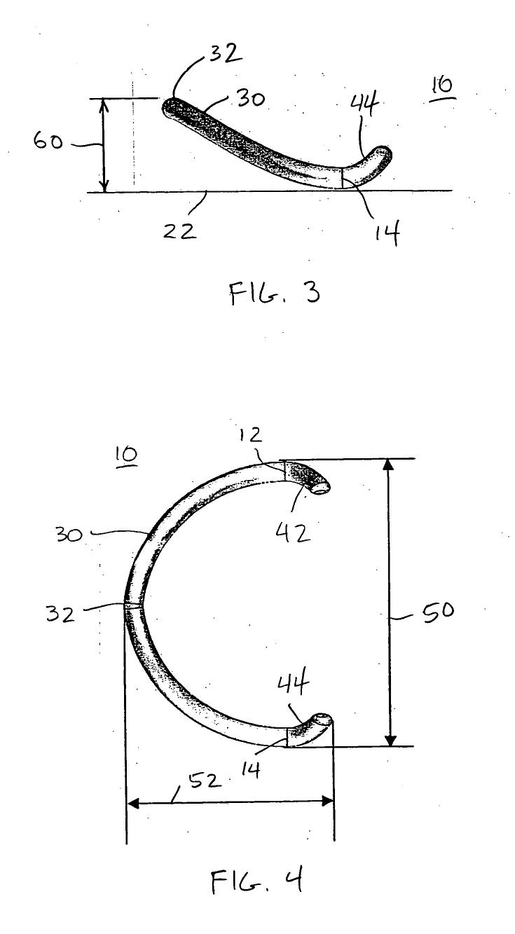

Prosthesis 10 is generally C shaped in plan view (see FIGS. 1 and 4). An upper medial point 12 and a lower medial point 14 of the C may be thought of as lying in a plan view plane of the prosthesis. This plan view plane is indicated by the (imaginary) line 20 in FIG. 2. Line 22 (also imaginary) in FIG. 3 also lies in this plane. Thus lines 20 and 22, which are perpendicular to one another, define the referenced plan view plane. Points 12 and 14 are referred to as medial, not because they are at the midpoint(s) of any structure, but only because they are interior to the length of the C-shaped structure (i.e., not at the free end points of the C).

The back 30 of the C is deflected upwardly out of the above-mentioned plan view plane (defined by lines 20 and 22 as described above). This upward deflection is clearly visible in FIGS. 2 and 3. It preferably starts at each of points 12 and 14, and also preferably goes smoothly up to a maximum upward deflection at a midpoint 32 on the back of the C between points 12 and 14. The shape of prosthesis 10 is preferably smooth at all points along the length of the prosthesis. The deflections described as upward in this specification will also be generally upward after prosthesis 10 has been implanted in a patient as a mitral valve prosthesis and the patient is standing upright.

The free end portions 42 and 44 of prosthesis 10 are also preferably deflected upwardly out of the above-mentioned plan view plane (defined by lines 20 and 22 as described above). Free end portion 42 is remote from back 30 beyond point 12 (i.e., free end portion 42 is on the opposite or far side of point 12 from back 30). Free end portion 44 is similarly remote from back 30 beyond point 14. Note that as is typical for a classic C shape, free end portions are synclinal (in plan view) in the direction of their free ends (see again FIGS. 1 and 4).

Points 12 and 14 are preferably at endpoints of a greatest height (or width) dimension 50 of prosthesis 10 (see FIG. 4). In use as a mitral valve prosthesis, points 12 and 14 are located at or near the commissures of the valve. Accordingly, dimension 50 may sometimes be referred to as the commissure-to-commissure (“CC”) dimension or the commissure width (“CW”) dimension of the prosthesis. FIG. 4 also shows the anterior-posterior (“AP”) dimension 52 of prosthesis 10.

The maximum upward deflection of back 30 is dimension 60 in FIGS. 2 and 3. Dimension 60 is preferably in the range from about 5% to about 25% of dimension 50. For example, dimension 60 may be in the range from about 3 mm to about 8 mm.

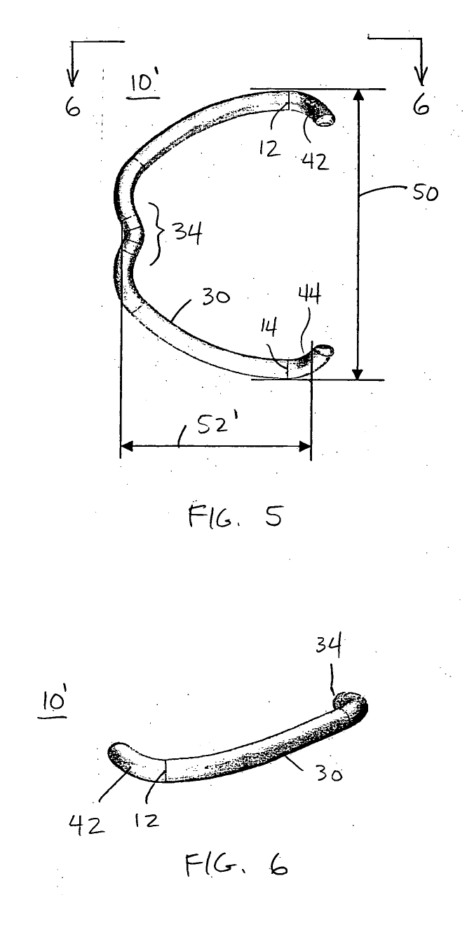

As shown in FIGS. 5 and 6, the back 30 of the prosthesis may have an indentation or pinch 34 to reduce the AP to CC ratio (i.e., the ratio of dimension 52′ to dimension 50). Pinch 34 is located at or near the center of back 30 of prosthesis 10′. Pinch 34 is inward, toward the open side of the C (corresponding, in use, to the anterior of the patient's mitral valve). In all other respects, prosthesis 10′ can be similar to prosthesis 10.

A prosthesis 10 or 10′ in accordance with this invention can be used for mitral valve repair by supporting the posterior section of the mitral annulus. The prosthesis is implanted, using techniques that can be conventional, with back 30 adjacent that posterior valve annulus section. The prosthesis aids in returning the posterior section of the mitral valve back to its natural saddle shape (commissures low and posterior and anterior sections arching upwardly between the commissures), and also provides support for a valve with functional mitral regurgitation.

Prosthesis 10 or 10′ is preferably fully rigid or at least semi-rigid to retain its saddle shape. As noted above, the saddle shape preferably has a 5% to 25% height-to-commissure-width ratio, or an absolute height from lowest point of the prosthesis to highest point of 3 mm to 8 mm.

To create a semi-rigid prosthesis 10 or 10′, the core material of the prosthesis can be made from a polymer such as ultra-high-molecular-weight polyethylene, polyurethane, ABS, or the like that will allow it to flex to some degree but that will also hold the saddle shape. Shape-memory alloys such as Nitinol can also be used to create such a semi-rigid prosthesis that flexes. A three-dimensional, semi-rigid prosthesis not only flexes in the X and Y directions (see (FIG. 1), but also in the Z direction (see FIG. 2). These three axes of flexibility will allow the ring to conform to the dynamic movement of the mitral valve region of the heart. Flexing of the prosthesis in the Z direction is accomplished to a large degree by wing flexing (indicated by arrows 70 in FIG. 2) of the prosthesis. The amount of flexing depends on the cross-sectional shape and elastic properties of the material employed for the prosthesis core. However, the amount of flexing employed should not allow the prosthesis to lose its saddle shape, unless there is an intentional purpose to do so. For example, the diameter of a wire, tube, or rod of shape-memory alloy used for the core will influence the amount of flex or movement that occurs after the material is formed into the shape desired.

A rigid prosthesis 10 or 10′ can be created using stronger material such as elgiloy, titanium, stainless steel, cobalt chrome, or ceramic. However, such a rigid prosthesis will not move with the heart in the same way as a semi-rigid prosthesis will.

Prosthesis 10 can have an anterior-posterior (“AP”) to commissure-commissure (“CC”) ratio in the range from about 0.75 to about 0.4 to treat most mitral valve diseases. (Again, dimensions 50 and 52 in FIG. 4 are the CC and AP dimensions, respectively.) To get the required amount of flexing in all three axes (X, Y and Z), the AP to CC ratio can vary within the 0.75 to 0.4 range.

By having a pinch 34 in the posterior section of prosthesis 10′ as shown in FIGS. 5 and 6, some sub-valvular remodeling of the left ventricle can additionally be created. Such a pinch 34 can have the effect of slowing down ischemia in the heart, and may repair or prevent other degenerative heart conditions.

It will be understood that the foregoing is only illustrative of the principles of the invention and that various modifications can be made by those skilled in the art without departing from the scope and spirit of the invention. For example, the principles of the invention may be applicable to prostheses for valves other than the mitral valve (e.g., the tricuspid valve). As another example of possible modifications within the scope of the invention, the illustrative embodiments shown herein are at least substantially symmetrical about a plane that (1) is perpendicular to the plan view plane, (2) passes through the high point 32 of back 30, and (3) passes midway between the free ends of the C. (This plane of symmetry can also be described as a plane perpendicular to a line between points 12 and 14, and which plane is midway between points 12 and 14.) However, such symmetry may not be desired in all cases, and it will be understood that various kinds of asymmetry can be employed to meet various needs.

1. A heart valve prosthesis comprising:
a structure, which in plan view is generally C-shaped, a medial upper point of the C and a medial lower point of the C lying in a plan view plane, a back of the C joining the upper and lower points and being deflected upwardly out of the plan view plane between the upper and lower points, and free end portions of the C away from the back beyond the upper and lower points also being deflected upwardly out of the plan view plane.

2. The prosthesis defined in claim 1 wherein the upper and lower points are endpoints of a greatest height of the C.

3. The prosthesis defined in claim 2 wherein the prosthesis is substantially symmetrical about a plane perpendicular to a line passing through the upper and lower points.

4. The prosthesis defined in claim 2 wherein the back has a maximum upward deflection out of the plan view plane that is in a range from about 5% to about 25% of the greatest height of the C.

5. The prosthesis defined in claim 2 wherein the back has a maximum upward deflection out of the plan view plane that is in a range from about 3 mm to about 8 mm.

6. The prosthesis defined in claim 1 wherein a central portion of the back is deflected toward an open side of the C.

7. The prosthesis defined in claim wherein the structure is substantially rigid.

8. The prosthesis defined in claim 1 wherein the structure is semi-rigid.
